(19) United States
(12) Patent Application Publication
(10) Pub. No.: US 2022/0362414 A1
(43) Pub. Date: Nov. 17, 2022

(54) SANITIZING FENDER FOR A WHEELCHAIR

(71) Applicants: Alfred Huber, Boca Raton, FL (US); Alan Jones, Boca Raton, FL (US)

(72) Inventors: Alfred Huber, Boca Raton, FL (US); Alan Jones, Boca Raton, FL (US)

(21) Appl. No.: 17/525,330

(22) Filed: Nov. 12, 2021

Related U.S. Application Data
(60) Provisional application No. 63/258,603, filed on May 17, 2021.

(51) Int. Cl. A61L 2/10; A61G 5/02; A61G 5/10

(52) U.S. Cl. CPC A61L 2/10; A61G 5/022; A61G 5/10; A61G 5/028; A61L 2202/16; A61L 2202/11

(57) ABSTRACT

A fender for sanitizing wheels of a wheelchair including a support frame and a fender housing. The support frame includes a mounting bracket for mounting the support frame on a vertical frame on the rear portion of the wheelchair. The fender housing is removably mounted on the support frame. The fender housing includes a plurality of Ultraviolet-C (UVC) light sources powered by a re-chargeable battery and a driver circuit. The plurality of UVC light sources are configured to sanitize a surface of a wheel of the wheelchair by virtue of illumination of surface of the wheel during rotation of the wheel.

### CROSS REFERENCE OF RELATED APPLICATIONS

This application claims the benefits of U.S. provisional application No. 63/258,603 filed May 17, 2021 entitled “Roll-O-Klenz” which provisional application is incorporated by reference herein in its entirety.

### TECHNICAL FIELD

The present invention relates to a fender and, particularly, to a sanitizing fender for a wheelchair.

### BACKGROUND

The object of the present invention is to provide a sanitizing fender for a wheelchair that solve problems in the prior art relating to issues faced by wheelchair bound patients in sanitizing the wheels of the wheelchair during motion over unclean and unprotected surfaces.

Typically, movement of conventional wheelchairs entail pushing of the wheels of the wheelchair by the patients. The pushing action is performed using hands. For an indisposed patient, this may involve risking the overall health as the hands may get contaminated based on contact with the wheels that themselves may be contaminated with virus, fungus, mold, protozoa, algae, and/or other harmful biological organisms. Further, the use of wheelchairs is high in medical facilities like hospitals, rehabilitation centers, and/or public places like airports, and the like. Consequently, due to high exposure of these places to pathogens and harmful organisms—even more so in ongoing times of a pandemic, the exposure of the patients using the wheelchair gets heightened exponentially.

Therefore there exists a need for providing a wheelchair with a mechanism that reduces the risk of patients getting infected due to cross contamination between wheels of the wheelchair and the hands.

### SUMMARY OF THE INVENTION

According to the embodiments illustrated herein, there is provided a fender for sanitizing wheels of a wheelchair including a support frame and a fender housing. The support frame includes a mounting bracket for mounting the support frame on a vertical frame on the rear section of the wheelchair. The fender housing may be removably mounted on the support frame. The fender housing includes a plurality of Ultraviolet-C (UVC) light sources powered by a re-chargeable battery and a driver circuit. The plurality of UVC light sources may be configured to sanitize a surface of a wheel of the wheelchair by virtue of illumination of surface of the wheel during rotation of the wheel.

In an embodiment, the support frame may be secured to the vertical frame by a mounting bracket clamp. The support frame in mounted position may be horizontally offset in a direction which is away from an armrest of the wheelchair.

In an embodiment, the fender housing may be mounted on the support frame using a plurality of screws passing through a plurality of slots present on the support frame. The position of the fender housing adjustable. The fender housing may be configured to be slid forward and backward.

In an embodiment, the re-chargeable battery may be charged using a Universal Serial Bus (USB) charging port. The USB charging port may be covered with a flap connected to the fender housing. The plurality of UVC light sources may be recessed into the fender housing. Further, the fender housing may include a switch for controlling the ON-OFF operation of the plurality of UVC light sources.

These features and advantages of the present disclosure may be appreciated by reviewing the following description of the present disclosure, along with the accompanying figures wherein like reference numerals refer to like parts.

### BRIEF DESCRIPTION OF DRAWINGS

The accompanying drawings illustrate the embodiments of systems, methods, and other aspects of the disclosure. Any person with ordinary skills in the art will appreciate that the illustrated element boundaries (e.g., boxes, groups of boxes, or other shapes) in the figures represent an example of the boundaries. In some examples, one element may be designed as multiple elements, or multiple elements may be designed as one element. In some examples, an element shown as an internal component of one element may be implemented as an external component in another, and vice versa. Furthermore, the elements may not be drawn to scale.

Various embodiments will hereinafter be described in accordance with the appended drawings, which are provided to illustrate, not limit, the scope, wherein similar designations denote similar elements, and in which:

FIG. 1 is a right-side elevation view of a mounted fender for sanitizing wheels of a wheelchair, in accordance with an embodiment of the present invention.

FIG. 2 is a right-side elevation view of a disengaged fender for sanitizing wheels of a wheelchair, in accordance with an embodiment of the present invention.

FIG. 3 is a rear section elevation view of the wheelchair having the fender mounted on the rear section, in accordance with an embodiment of the present invention.

FIG. 4 is a right-side elevation view of a mounted fender for sanitizing wheels of a wheelchair, in accordance with an embodiment of the present invention.

FIG. 5 is a perspective view of fender mounted on a wheel of a wheelchair, in accordance with an embodiment of the present invention.

FIG. 6 depicts a perspective view of the mounting brackets of the fender, in accordance with an embodiment of the present invention.

FIG. 7 depicts a perspective view of the fender housing, in accordance with an embodiment of the present invention.

FIG. 8 depicts a perspective view of the fender housing having a switch for operating the light sources, in accordance with an embodiment of the present invention.

FIG. 9 depicts a perspective view of the fender housing having a power supply for operating the plurality of UVC light sources, in accordance with an embodiment of the present invention.

### DETAILED DESCRIPTION

The present disclosure is best understood with reference to the detailed figures and description set forth herein. Various embodiments have been discussed with reference to the figures. However, those skilled in the art will readily appreciate that the detailed descriptions provided herein with respect to the figures are merely for explanatory purposes, as the methods and systems may extend beyond the described embodiments. For instance, the teachings presented and the needs of a particular application may yield multiple alternative and suitable approaches to implement the functionality of any detail described herein. Therefore, any approach may extend beyond certain implementation choices in the following embodiments.

References to “one embodiment”, “at least one embodiment”, “an embodiment”, “one example”, “an example”, “for example”, and so on indicate that the embodiment(s) or example(s) may include a particular feature, structure, characteristic, property, element, or limitation, but not every embodiment or example necessarily includes that particular feature, structure, characteristic, property, element, or limitation. Furthermore, repeated use of the phrase “in an embodiment” does not necessarily refer to the same embodiment.

Unless defined otherwise, all technical and scientific terms used herein have the same meaning as commonly understood by one of the ordinary skills in the art to which this invention belongs. Although any method and material similar or equivalent to those described herein can also be used in the practice or testing of the present invention, the preferred methods and materials have been described. All publications, patents, and patent applications mentioned herein are incorporated in their entirety.

It is noted that as used herein and in the appended claims, the singular forms “a”, “and”, and “the” include plural referents, unless the context clearly dictates otherwise. In the claims, the terms “first”, “second”, and so forth are to be interpreted merely as ordinal designations; they shall not be limited in themselves. Furthermore, the use of exclusive terminology such as “solely”, “only”, and the like in connection with the recitation of any claim element is contemplated. It is also contemplated that any element indicated to be optional herein may be specifically excluded from a given claim by way of a “negative” limitation. Finally, it is contemplated that any optional feature of the inventive variation(s) described herein may be set forth and claimed independently or in combination with any one or more of the features described herein.

All references cited herein, including publications, patent applications, and patents, are hereby incorporated by reference to the same extent as if each reference were individually and specifically indicated to be incorporated by reference, and were set forth in its entirety herein.

The recitation of ranges of values herein is merely intended to serve as a shorthand method of referring individually to each separate value falling within the range, unless otherwise indicated herein, and each separate value is incorporated into the specification as if it were individually recited herein.

The disclosed and claimed invention herein provides a fender for sanitizing wheels of a wheelchair including a support frame and a fender housing. The support frame includes a mounting bracket for mounting the support frame on a vertical frame on the rear section of the wheelchair. The fender housing may be removably mounted on the support frame. The fender housing includes a plurality of Ultraviolet-C (UVC) light sources powered by a re-chargeable battery and a driver circuit. The plurality of UVC light sources may be configured to sanitize a surface of a wheel of the wheelchair by virtue of illumination of surface of the wheel during rotation of the wheel.

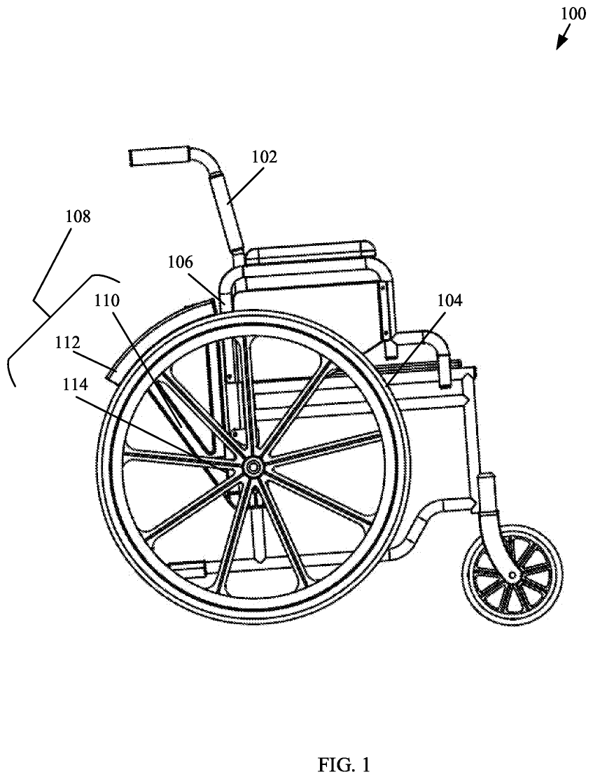

FIG. 1 is a right-side elevation view of a mounted fender for sanitizing wheels of a wheelchair, in accordance with an embodiment of the present invention. With regards to the FIG. 1, there is shown a right-side elevation view 100 of a wheelchair 102 that is configured to accommodate the fender 108. For the purpose of illustration, the wheelchair 102 includes a wheel 104 and a rear section 106.

FIG. 1 depicts a plurality of components of the fender 108 mounted on the wheelchair 102. For the purposes of illustration, only a single side of the wheelchair 102 has been depicted. A person of ordinary skill in the art will appreciate that the similar assembly may be mounted on the left side of the wheelchair 102, without limiting the scope of the disclosure.

In an embodiment, the fender 108 may include a support frame 110 and a fender housing 112. The fender housing 112 may be removably mounted on the support frame 110. As is depicted, while in a mounted position, the support frame 110 of the fender 108 is horizontally offset in a direction, which is away from an armrest of the wheelchair. In case of the depicted fender 108, the fender can be seen to be attached to the rear section 106 in a manner that the fender 108 appears to be jutting out from the rear section 106. Further, the fender 108 in mounted position is such that the fender housing 112 is placed 1-2 inches above the wheel 104.

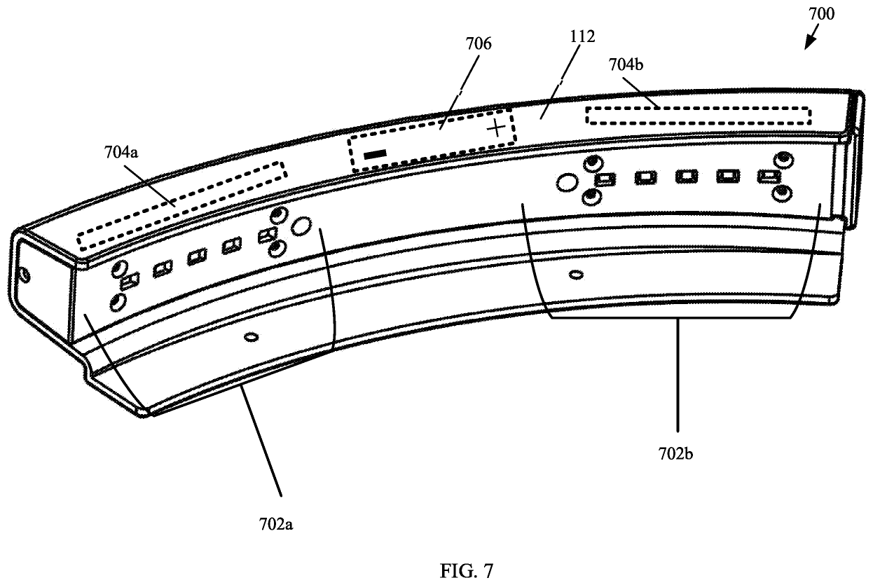

In an embodiment, the fender housing 112 includes a plurality of Ultraviolet-C (UVC) light sources powered by a re-chargeable battery and a driver circuit (depicted in FIG. 7). In an embodiment, the re-chargeable battery may include, but is not limited to, Lithium-Ion (Li-ion) battery, Nickel-Cadmium (Ni—Cd) battery, Nickel Metal Hydride (Ni-MH), and the like. The plurality of UVC light sources may be recessed into the fender housing. In an embodiment, the re-chargeable battery may be charged using a Universal Serial Bus (USB) charging port. Further, the USB charging port may be covered with a flap connected to the fender housing 112.

A person of ordinary skill in the art will appreciate that the charging of the re-chargeable battery may be based on one or more other charging mechanisms, such as, lightning conductor, USB-C connector, wireless charging, and the like, without limiting the scope of the disclosure.

In an embodiment, the surface of the fender housing 112 that is proximal to the armrest of the wheelchair 102 may include a switch for controlling the ON-OFF operation of the plurality of UVC light sources. In another embodiment, the switch ON-OFF operation may be controlled based on one or more image sensors configured to detect one or more pre-defined gestures of the patient on the wheelchair 102.

When switched ON, the plurality of UVC light sources may illuminate the wheel 104 of the wheelchair 102 in a direction, which faces the surface on which the wheelchair 102 is configured to move. Consequently, the plurality of UVC light sources may be configured to sanitize a surface of a wheel 104 of the wheelchair 102 by virtue of illumination of surface of the wheel 104 during rotation of the wheel. In an embodiment, the plurality of UVC light sources may include, but are not limited to, medical grade UVC Light Emitting Diode (LED), one or strips of UVC LEDs, UVC bulbs, and the like.

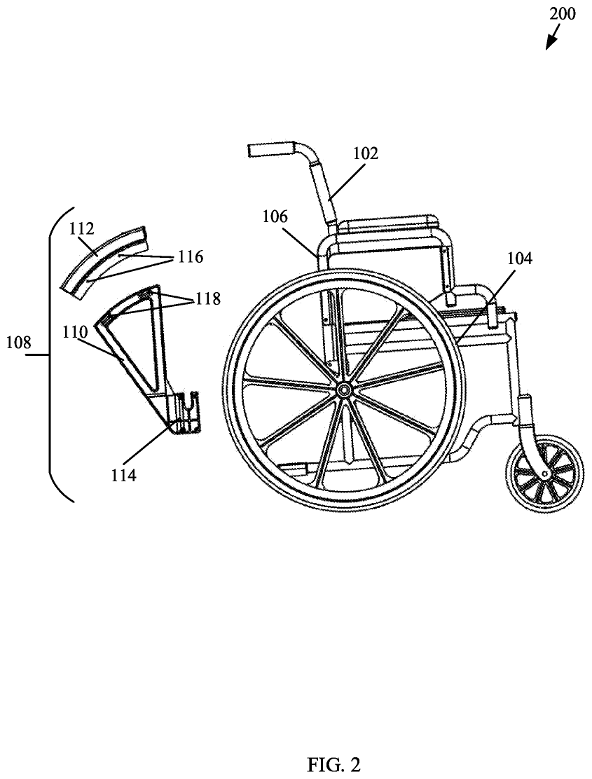

FIG. 2 is a right-side elevation view of a disengaged fender for sanitizing wheels of a wheelchair, in accordance with an embodiment of the present invention. Elements of FIG. 2 have been explained in conjunction with the elements depicted in FIG. 1.

With regards to the FIG. 2, there is shown a right-side elevation view 200 of a wheelchair 102 that is configured to accommodate the fender 108. When compared to FIG. 1, FIG. 2 depicts the fender 108 in a disengaged position with respect to the wheelchair 102. Furthermore, the components of the fender 108 that include support frame 110 and the fender housing 112 have been depicted in a disengaged position with respect to each other.

FIG. 2 further depicts the mechanism using which the fender housing 112 is mounted on the support frame 110. In an embodiment, the mechanism may be based a plurality of screws 116 passing through the plurality of slits 118 provided on the support frame 110. The plurality of slits 118 are such that even after the fender housing 112 may be screwed to the support frame, the fender housing may be slid in a forward and backward direction to facilitate adjustment of the fender housing 112 with respect to the position of the armrest of the wheelchair 102.

In an alternate embodiment, the fender housing 112 may be secured to the support frame 110 using mechanisms for attaching that include, but are not limited to, magnet based attachment, slot and notch mechanism, and the like, without limiting the scope of the disclosure.

FIG. 2 further depicts a bracket clamp 114 using which the support frame 110 may be mounted on the axle of the wheelchair 102 present in the bottom rear section 106. In an embodiment, the bracket clamp 114 may include a plurality of components having a male member and a female member. The plurality of components may be bind together from both the sides of the wheel 104, in order to secure the support frame 110 in fixed position when mounted. Furthermore, the bracket clamp 114 may be a U-shaped clamp that may be intended to prevent the rotation of the support frame 110 about the axle of the wheelchair 102.

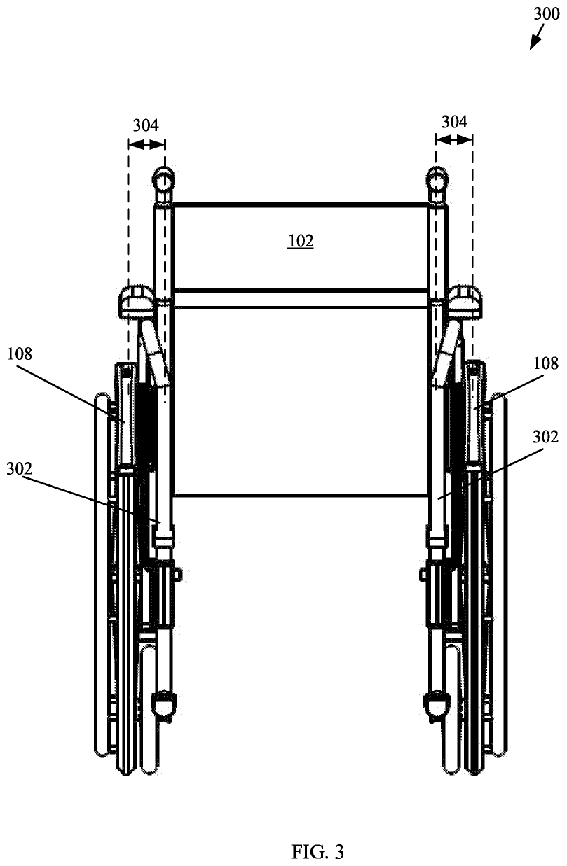

FIG. 3 is a rear section elevation view 300 of the wheelchair having the fender mounted on the rear section 106, in accordance with an embodiment of the present invention. Elements of FIG. 3 have been explained in conjunction with the elements depicted in FIGS. 1 to 2. With reference to FIG. 3, there is depicted the rear section 106 of the wheelchair 102. The wheelchair includes a plurality of armrest 302.

FIG. 3 further illustrates the offset that is present between the each of the plurality of armrest 302 and the fender 108, when the fender 108 is mounted on the wheelchair 102. The offsets have been depicted as gaps 304. The gaps 304 facilitate free movement of the plurality of armrest 302 whose position may be manipulated in forward or backward directions without being obstructed by the mounted fenders 108.

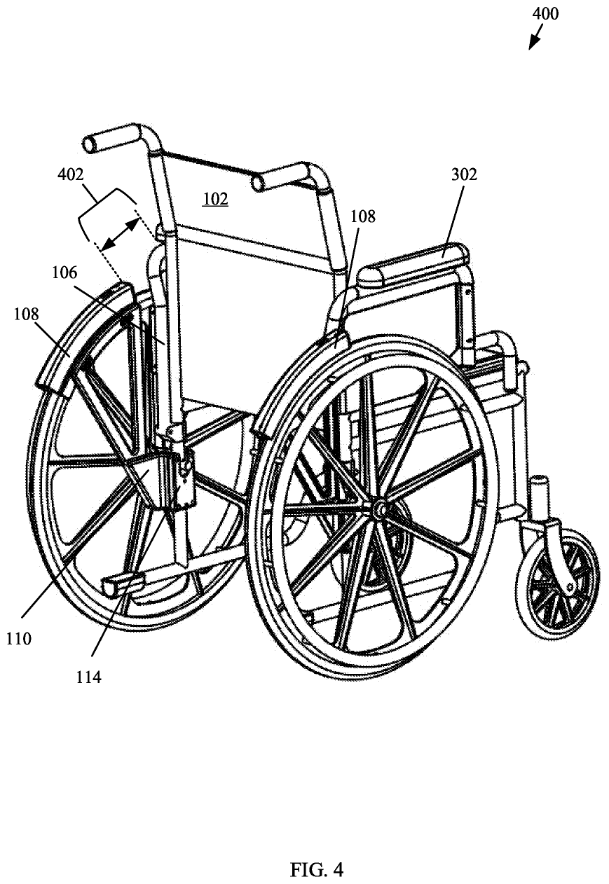

FIG. 4 is a right-side elevation view 400 of a mounted fender for sanitizing wheels of a wheelchair, in accordance with an embodiment of the present invention. Elements of FIG. 4 have been explained in conjunction with the elements depicted in FIGS. 1 to 3. With reference to FIG. 4, there is depicted the rear section 106 of the wheelchair 102.

The wheelchair 102 the fenders 108 in a mounted position. FIG. 4 further depicts the bracket clamp 114 that is used for fixing the support frame 110 to the rear section 106 of the wheelchair 102. The support frame 110 is constructed in a manner that the fender 108 in a mounted position is offset from the armrest 302 by a gap 402 such that the fender 108 is behind the armrest 302.

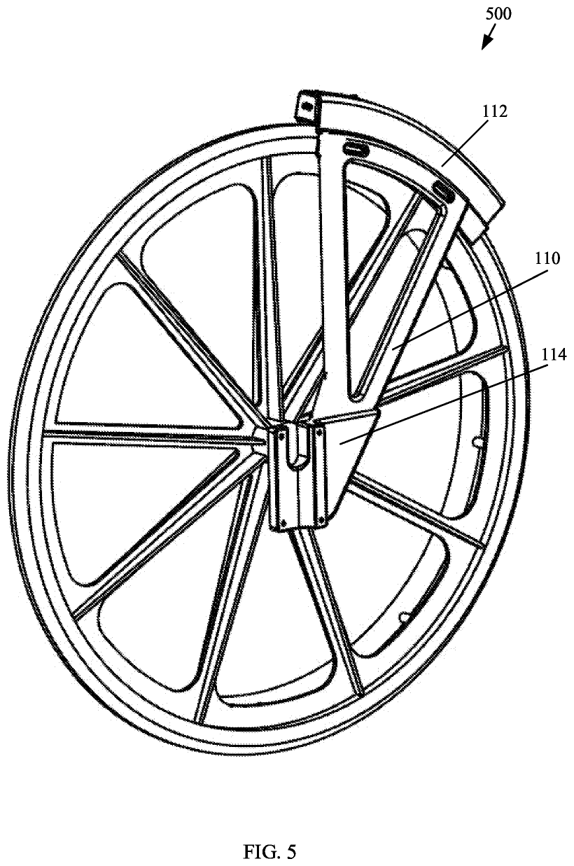

FIG. 5 is a perspective view 500 of the fender mounted on a wheel of a wheelchair, in accordance with an embodiment of the present invention. Elements of FIG. 5 have been explained in conjunction with the elements depicted in FIGS. 1 to 4. With reference to FIG. 5, there is depicted a wheel 104 of the wheelchair 102. Attached to the axle of the wheel 104 is the fender 108. It is further illustrated from FIG. 5 how the base of the triangular structure of the support frame 110 leads to creation of an offset between the fender housing 112 and the armrest of the wheelchair 102.

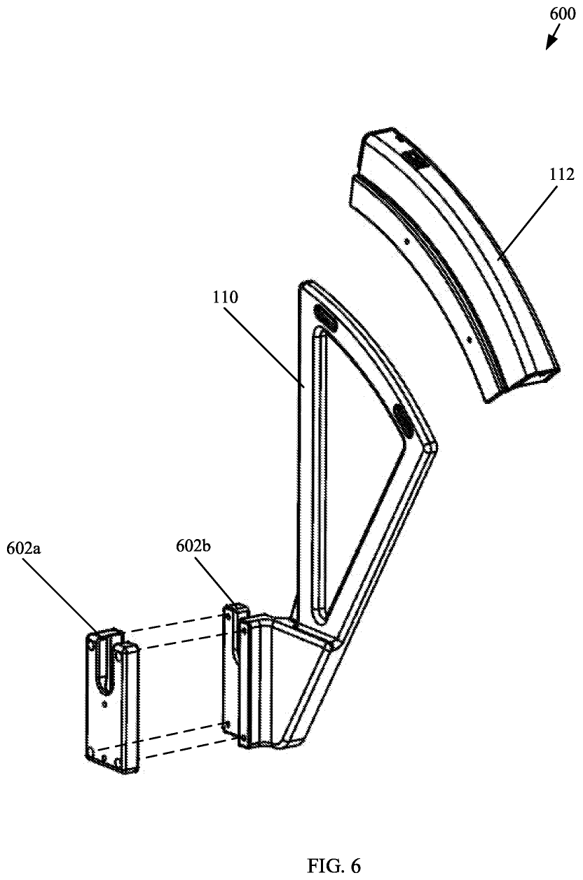

FIG. 6 depicts a perspective view 600 of the mounting bracket of the fender, in accordance with an embodiment of the present invention. Elements of FIG. 6 have been explained in conjunction with the elements depicted in FIGS. 1 to 5.

The view depicts the fender 108 along with the components of the support frame 110 and the fender housing 112. In an embodiment, the support frame includes a plurality of mounting brackets 602 using which the support frame 110 is mounted on the rear section 106 of the wheelchair. The plurality of mounting bracket 602 include bracket 602a and bracket 602b. For mounting the support frame 110, the brackets 602a and 602b are aligned with each other and then fixed to each other using a plurality of screws. A person of ordinary skill in the art will appreciate that any other mechanism for affixing the plurality of brackets 602 may be used, without limiting the scope of the disclosure. The plurality of brackets 602 prevent the mounted support frame 110 from rotation.

FIG. 7 depicts a perspective view 700 of the fender housing, in accordance with an embodiment of the present invention. Elements of FIG. 6 have been explained in conjunction with the elements depicted in FIGS. 1 to 6.

The view depicts the fender housing 112 of the fender 108 along with the components that may be integrated inside the fender housing 112. In an embodiment, the fender housing may include a plurality of UVC light sources 702a and 702b. The plurality of UVC light sources may include, but are not limited to, medical grade UVC Light Emitting Diode (LED), one or strips of UVC LEDs, UVC bulbs, and the like. The operation of the plurality of UVC light sources 702a and 702b may be based on a plurality of driver circuits 704a and 704b, respectively.

In an embodiment, the plurality of driver circuits (704a and 704b) may be based on printed circuit board (PCB) configured with electronic components for operating the plurality of UVC light sources 702a and 702b. In an embodiment, the driver circuit may include a microcontroller that may enable controlling the operation of the light sources using a mobile device coupled with the driver circuit.

The power supply to the plurality of driver circuits 704a and 704b may be provided by a re-chargeable battery 706. In an embodiment, the fender housing may include a dedicated portion for accommodating the re-chargeable battery 706. In an embodiment, the re-chargeable battery 706 may include, but is not limited to, Lithium-Ion (Li-ion) battery, Nickel-Cadmium (Ni—Cd) battery, Nickel Metal Hydride (Ni-MH). The fender housing 112 may further include a provision for charging the re-chargeable battery 706. In an embodiment, the re-chargeable battery 706 may be charged using a USB charging port. Further, the USB charging port may be covered with a flap connected to the fender housing 112.

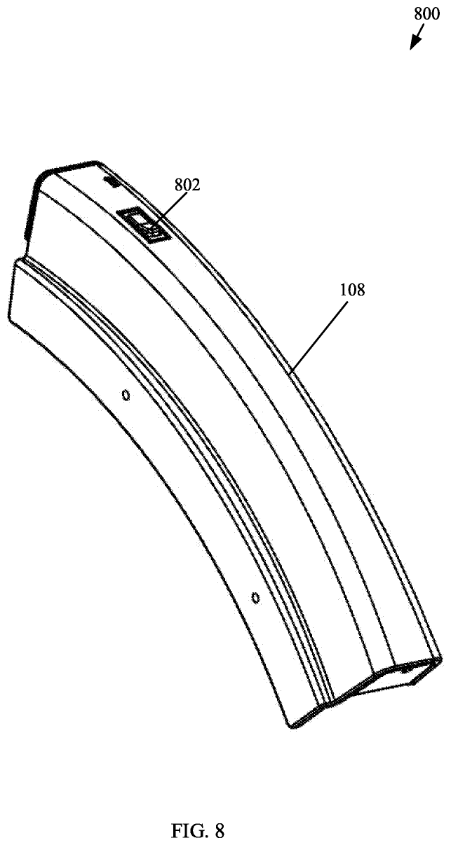

FIG. 8 depicts a perspective view 800 of the fender housing having a switch for operating the light sources, in accordance with an embodiment of the present invention. Elements of FIG. 8 have been explained in conjunction with the elements depicted in FIGS. 1 to 7.

In an embodiment, the fender housing 112 may include a switch 802 on the surface of the fender housing 112 that is proximal to the armrest of the wheelchair 102. The switch may facilitate controlling ON-OFF operation of the plurality of UVC light sources 702a and 702b.

A person of ordinary skill in the art will appreciate that in an embodiment, a sensor may be provided in place of the switch. The sensor may be used for controlling the ON-OFF operation. Such an operation may be based on one or more image sensors configured to detect one or more pre-defined gestures of the patient on the wheelchair 102.

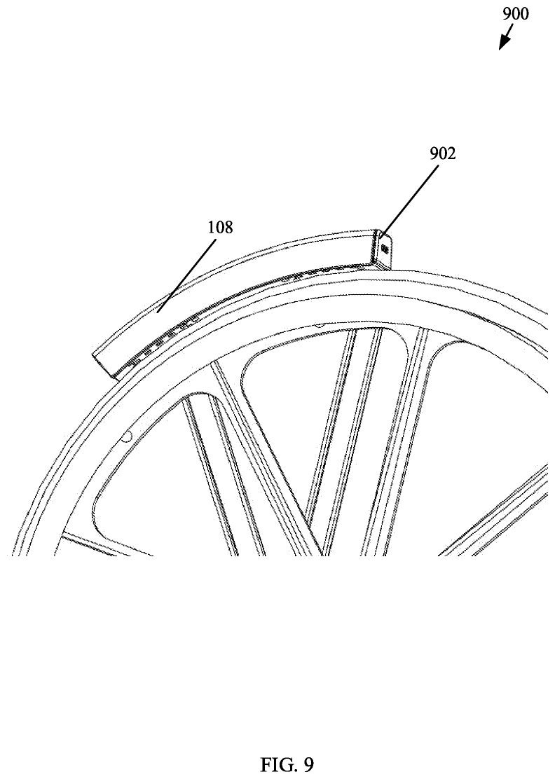

FIG. 9 depicts a perspective view 900 of the fender housing having a power supply for operating the plurality of UVC light sources 702a and 702b, in accordance with an embodiment of the present invention. Elements of FIG. 9 have been explained in conjunction with the elements depicted in FIGS. 1 to 8.

With respect to FIG. 9, there is shown the fender 108 mounted on the wheelchair 102. As explained in FIG. 7, the fender housing 112 includes the plurality of UVC light sources 702a and 702b powered by a re-chargeable battery 706. The battery may be recharged via the USB charging port 902. In an embodiment, the USB charging port 902 may be covered with a flap connected to the fender housing 112.

While the summary and detailed description show and describe preferred embodiments of the present invention, those skilled in the art recognize those embodiments as examples. The specification does not limit the invention by the specific examples provided. Instead, those skilled in the art recognize variations, changes, and substitutions without departing from the invention. Furthermore, all aspects of the invention are not limited to the specific depictions, configurations, or relative proportions set forth herein which depend upon a variety of conditions and variables. The inventor contemplates that the invention shall cover alternatives, modifications, variations, or equivalents.

### Advantages

The disclosed shoe with a fender for sanitizing wheels of a wheelchair allows for increased hygiene and cleanliness while using the wheelchair. The fender also facilitates decreasing the spread of viruses and bacteria as the wheels remain sanitized by action of UVC light source on possibly contaminated wheels of the wheelchair. This further leads to added safety and freedom from repeated sanitizing of the hands while using the wheelchair. This is additionally immensely beneficial in home care setups as well because the patients and elderly may remain free from the need for having a dedicated nursing staff for taking them from one place to another on the wheelchair. Knowing the fact that the wheels are sanitized, they may perform the movement all by themselves without the risk of being infected through cross-contamination between their hands and the wheels.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise. The terms “including”, “comprising”, “having” and variations thereof mean “including but not limited to”, unless expressly specified otherwise. The terms “a”, “an” and “the” mean “one or more”, unless expressly specified otherwise.

No language in the specification should be construed as indicating any non-claimed element as essential to the practice of the invention. A description of an embodiment with several components in communication with each other does not imply that all such components are required. On the contrary, a variety of optional components are described to illustrate the wide variety of possible embodiments of the invention.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

A person with ordinary skills in the art will appreciate that the systems, components, and sub-components have been illustrated and explained to serve as examples and should not be considered limiting in any manner. It will be further appreciated that the variants of the above disclosed system elements, components, and sub-components, and other features and functions, or alternatives thereof, may be combined to create other different systems or applications.

Those skilled in the art will appreciate that any of the aforementioned steps and/or system modules may be suitably replaced, reordered, or removed, and additional steps and/or elements, components, and sub-components may be inserted, depending on the needs of a particular application.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

We claim:

1. A fender for sanitizing wheels of a wheelchair, the fender comprising:
a support frame comprising a mounting bracket for mounting the support frame on a vertical frame on a rear section of the wheelchair; and
a fender housing removably mounted on the support frame,
wherein the fender housing comprises a plurality of Ultraviolet-C (UVC) light sources powered by a re-chargeable battery and a driver circuit; and
wherein the plurality of UVC light sources are configured to sanitize a surface of a wheel of the wheelchair by virtue of illumination of the surface of the wheel during rotation of the wheel.

2. The fender as claimed in claim 1, wherein the support frame is secured to the vertical frame by a mounting bracket clamp.

3. The fender as claimed in claim 1, wherein the fender housing is mounted on the support frame using a plurality of screws passing through a plurality of slots present on the support frame.

4. The fender as claimed in claim 3, wherein a position of the fender housing is adjustable, and wherein the fender housing is configured to be slid forward and backward.

5. The fender as claimed in claim 1, wherein the re-chargeable battery is charged using a Universal Serial Bus (USB) charging port.

6. The fender as claimed in claim 5, wherein the USB charging port is covered with a flap connected to the fender housing.

7. The fender as claimed in claim 1, wherein the plurality of UVC light sources are recessed into the fender housing.

8. The fender as claimed in claim 1, wherein the fender housing comprises a switch for controlling an ON-OFF operation of the plurality of UVC light sources.

9. The fender as claimed in claim 1, wherein the support frame in mounted position is horizontally offset in a direction which is away from an armrest of the wheelchair.
